(19) United States
(12) Patent Application Publication
(10) Pub. No.: US 2024/0423901 A1
(43) Pub. Date: Dec. 26, 2024

(54) COSMETIC COMPOSITION COMPRISING SUBCRITICAL WATER EXTRACT OF PLASMA-TREATED CAVIAR

(71) Applicants: COSMAX, INC., Hwaseong-si (KR); ALMASCAVIAR CO., LTD., Hwaseong-si (KR)

(72) Inventors: Yae Rin LEE, Anyang-si (KR); Sang Hun HAN, Hwaseong-si (KR); Je Hee HAN, Hwaseong-si (KR); Kyung Eun LEE, Seongnam-si (KR); Su Gyeong JEONG, Seoul (KR); Seok Kyun YUN, Seongnam-si (KR); Seung Hyun KANG, Seoul (KR); Myeong Sam PARK, Seoul (KR)

(73) Assignees: COSMAX, INC., Hwaseong-si (KR); ALMASCAVIAR CO., LTD., Hwaseong-si (KR)

(21) Appl. No.: 18/692,448

(22) PCT Filed: Sep. 7, 2022
(86) PCT No.: PCT/KR2022/013466

§ 371 Date: Mar. 15, 2024

(30) Foreign Application Priority Data
Sep. 15, 2021 (KR) 10-2021-0123168

(51) Int. Cl. A61K 8/98; A61Q 19/08

(52) U.S. Cl. CPC A61K 8/987; A61Q 19/08; A61K 2800/81

(57) ABSTRACT

The present invention relates to a cosmetic composition comprising a subcritical water extract of plasma-treated caviar, which can be usefully used for the prevention, amelioration, or treatment of skin-related conditions, such as skin wrinkle improvement, elasticity enhancement, anti-aging, or the like.

### TECHNICAL FIELD

The present application claims priority to Korean Patent Application No. 10-2021-0123168, filed on Sep. 15, 2021, which is hereby incorporated by reference in its entirety.

The present application relates to a cosmetic composition.

### REFERENCE TO SEQUENCE LISTING SUBMITTED ELECTRONICALLY

This application contains a sequence listing, which is submitted electronically as an XML formatted sequence listing with a file name “688942_1241_Sequence_Listing”, creation date of Mar. 15, 2024, and having a size of 6,388 bytes. The sequence listing submitted electronically is part of the specification and is herein incorporated by reference in its entirety.

### BACKGROUND ART

As the human body ages, it undergoes physiological and physical aging. Among them, a typical symptom of aging skin is the development of wrinkles, which causes the skin to atrophy, lose elasticity, and become dry. Skin aging is a phenomenon caused by structural and functional changes in skin cells and tissues, and its causes may be broadly divided into intrinsic aging and extrinsic aging. Intrinsic aging is an inevitable process that occurs as we age, causing wrinkles, loss of skin elasticity, loss of moisture in skin cells, and changes in the structure of the stratum corneum. On the other hand, extrinsic aging is caused by external stress such as environmental factors, of which ultraviolet radiation is the main cause, and is also called photoaging.

In aged skin, the ability to produce collagen (COL1A1; alpha-1 type I collagen) and elastin decreases, while proteolytic enzymes (MMP; matrix metalloproteinase, collagenase 1) increase, breaking down the collagen that supports and fills our skin, damaging the skin and contributing to loss of elasticity and wrinkles. As a result, proteolytic enzymes break down collagen and elastin, and aged skin gradually becomes thinner and tension decreases, making the skin weak and soft.

Caviar is the roe of a sturgeon, and salted caviar food is one of the world's top three delicacies and is well known as a luxurious food ingredient. Caviar is composed of various nutrients such as protein, fat, sugar, and vitamins, and is reported to be a perfect nutritious food with a particularly high protein content. In particular, caviar is rich in essential amino acids such as leucine, arginine, lysine, and isoleucine, as well as various amino acids such as glutamic acid, serine, and alanine, and is characterized by being absorbed deep into the skin. In addition, the fat contained in caviar is mostly composed of cholesterol and lecithin, which acts to regenerate the lipid membrane of the epidermis, and the rich omega-3 fatty acids show an anti-aging effect. Therefore, caviar is included as a luxurious ingredient in some cosmetics and the like.

In general, thermal water, organic solvent, or supercritical extraction methods are used to extract active ingredients from natural products. In particular, in the fields of food and cosmetics, it is known that water or organic solvents such as alcohol and supercritical carbon dioxide are used as extraction solvents due to the stability of residual solvent with respect to the human body. However, since hot water extraction uses high-temperature water, hot water extraction is mostly used for extraction of polar substances, and since this method is performed at a high temperature, there are problems in that the substance to be extracted is denatured by heat and that the extract quality deteriorates due to inevitable contact with oxygen in the air during the hot water extraction process.

In addition, in the case of extraction technology using organic solvents, which is commonly used for extraction of natural products, there are a lot of impurities mixed in, and there is the inconvenience of having to remove the residual solvent, which is a harmful substance, and not only is the process complicated because the extraction technology using organic solvent goes through several processes, but there are also problems such as the possibility of foreign substances being mixed in with the organic solvent used and the low purity of the extract.

Therefore, there is a need to develop a method of extracting ingredients derived from natural products which may be useful for skin-related conditions.

### DISCLOSURE

### Technical Problem

One aspect relates to providing a cosmetic composition for improving skin condition including subcritical water extract of plasma-treated caviar as an active ingredient.

One aspect is to provide a method of preparing a subcritical water extract of plasma-treated caviar.

### Technical Solution

One aspect provides a composition for improving skin condition including subcritical water extract of plasma-treated caviar as an active ingredient. In an embodiment, the extract may be a subcritical water extract of low temperature/atmospheric-pressure plasma-treated caviar.

As used herein, the term “low temperature/atmospheric-pressure plasma” has the advantage of being highly chemically reactive to the target object without thermal change, being energy stable, and not altering or damaging the state of the interacting substances because the low temperature/atmospheric-pressure plasma only acts on the surface of the reacting substances. Specifically, low temperature/atmospheric-pressure plasma may be generated by dielectric barrier discharge, corona discharge, microwave discharge, arc discharge, or air discharge. For example, an apparatus that generates low temperature/atmospheric-pressure plasma may be a dielectric barrier discharge (DBD) method using AC power, where the base electrode is located inside a tube made of teflon, and another electrode surrounded by teflon is located at the center of the tube. Discharge occurs in the space between the two electrodes, and the gap between the two cylinders is 1 mm to 10 mm. The cylindrical discharge space is designed to allow the passage of discharge-driving gas such as argon, etc. An AC power is applied to the electrode located in the center, the frequency is 5 kHz to 350 kHz, and the voltage is rms value of 10 or more. In addition, the plasma flame size of the apparatus generating the low temperature/atmospheric-pressure plasma may be up to 10 mm, and the driving gas inflow rate may be set to 5 L/min or more. The driving gas may be an inert gas that does not affect the caviar component, for example, argon, oxygen, helium gas, etc., and may be set to a frequency of 20 kHz to 50 kHz, an input voltage of 5 V to 10 V, and output voltage 10 W to 30 W. In an embodiment, the low temperature/atmospheric-pressure plasma may be performed under conditions of a temperature of 10° C. to 80° C. and a pressure of 0.5 atmospheres (atm) to 10 atm. The low temperature/atmospheric-pressure plasma may be performed at a temperature of, for example, 10° C. to 80° C., 10° C. to 70° C., 10° C. to 50° C., 20° C. to 60° C., 30° C. to 50° C., or 40° C. to 80° C. At this time, if the plasma operating temperature is less than the above range or greater than the above range, not only is it difficult to secure various applicability and stability, but there are problems in that excessive cost is invested and the process becomes difficult. Additionally, the low temperature/atmospheric-pressure plasma may be performed at a pressure of, for example, 0.5 atm to 10 atm, 1 atm to 10 atm, 1 atm to 8 atm, 2 atm to 7 atm, 3 atm to 9 atm, or 1 atm to 5 atm. At this time, if the plasma operating pressure is less than the above range or greater than the above range, there are limitations such as having to manufacture a separate vacuum vessel for plasma processing and installing a vacuum pump to maintain vacuum, which reduces the economic feasibility and efficiency of equipment management and operation costs. Therefore, the caviar according to an aspect is plasma-treated under the above temperature and pressure conditions, so that the active ingredients of the caviar may be extracted with high efficiency and high purity.

The plasma exposure time refers to the exposure time for a single plasma-treatment of the lyophilized caviar powder, and the number of plasma exposures refers to the number of repeated exposures to plasma. In an embodiment, the plasma exposure time may be from 1 minute to 3 minutes per treatment, and the number of repeated exposures may be from 2 times to 10 times for 1 minute to 2 minutes every 30 minutes, or from 2 times to 5 times for 2 minutes every hour.

As used herein, the term “subcritical water extraction” refers to a method of decomposition and extraction of substances using ionized water under conditions of temperature and pressure below the critical point of water (374° C., 22.1 MPa), also known as pressurized hot water extraction (PHWE) or pressurized low polarity water extraction (PLPW). The advantage of this subcritical water extraction is that the solvent used is water, the extraction time is short, about 5 minutes to 30 minutes, and it is known to be efficient in extracting non-polar substances such as phenol compounds. The subcritical water extraction may be performed at a temperature of 40° C. to 120° C. For example, the subcritical water extraction may be performed at a temperature of 40° C. to 120° C., 40° C. to 100° C., 40° C. to 80° C., or 80° C. to 120° C. At this time, if the subcritical water extraction temperature is less than the above range, there is a problem that the yield of caviar extract is reduced, and if the subcritical water extraction temperature is greater than the above range, there is a problem that the stability of the active ingredient is reduced. Additionally, the extraction pressure may be performed at a pressure of 0.5 atm to 10 atm. The subcritical water extraction may be performed at a pressure of, for example, 0.5 atm to 10 atm, 0.5 atm to 8 atm, 1 atm to 7 atm, 3 atm to 6 atm, or 4.5 atm to 5.5 atm. In this case, if the subcritical water extraction pressure is less than the above range, there is a problem that the vaporization point is reduced and the extraction efficiency of the active ingredient is reduced, and if the subcritical water extraction pressure is greater than the above range, there is a problem that the stability of the active ingredient is reduced.

In the Example, it was confirmed that the caviar extract extracted by subcritical water extraction after low temperature and atmospheric-pressure plasma-treatment increased the content of anti-aging and anti-wrinkle factors, for example COL1A1, and/or decreased the expression of matrix metalloproteinases-1 (MMPs-1), a matrix proteolytic enzyme. Therefore, the composition including caviar low temperature and atmospheric-pressure plasma and subcritical water extract induces collagen synthesis in the skin by increasing the expression of COL1A1 and inhibits the decomposition of collagen and elastin by inhibiting the expression of MMP-1, resulting in improved skin beauty, for example, the composition may have the effect of suppressing skin aging, strengthening the skin barrier, suppressing skin wrinkles, or moisturizing the skin.

In an embodiment, the composition may be used to improve skin condition or improve skin beauty. For example, the skin condition or skin beauty improvement may be inhibiting or improving skin aging, anti-wrinkle, skin elasticity, skin regeneration, skin barrier strengthening, or skin moisturizing.

As used herein, the term “skin aging” refers to the tangible and intangible changes that occur in the skin with age, for example thinning of the epidermis, decrease in, the number of cells and blood vessels in the dermis, the ability to repair DNA damage, the cell turnover cycle, wound healing, skin barrier function, epidermal water retention, sweating, sebum secretion, vitamin D production, physical damage defense, chemical removal, immune response, sensory function, and thermoregulation. In particular, wrinkles and loss of elasticity are a major symptom of skin aging, which is believed to be caused by a decrease in collagen and elastin, the main proteins in the dermis, and a modification of the network of these elastic fibers. The dense network of elastin and fibronectin in the dermis and epidermis maintains the cushioning layer of the skin, but as the number of collagen fibers decreases with aging, the network density of the elastic fibers decreases as they fragment and twist, resulting in a loss of normal skin support.

The composition may be used to improve skin aging caused by extrinsic or intrinsic factors. The exogenous factors refer to various external factors, such as ultraviolet radiation (light), and endogenous factors are also referred to as chronological factors, and mainly refer to factors caused by the passage of time. In other words, the skin aging specifically includes not only premature aging symptoms induced by external stimuli such as ultraviolet rays, pollution, cigarette smoke, chemicals, etc., but also is a concept that includes the natural aging phenomenon that occurs as skin cell proliferation decreases with age, and includes wrinkles, loss of elasticity, skin sagging, and dryness. In addition, wrinkles include stimulation caused by changes in internal and external factors that change the components that make up skin tissue, causing wrinkles.

The aging may be photoaging. As used herein, the term “photoaging” refers to a phenomenon induced by external environmental factors, the most representative of which is ultraviolet radiation. Ultraviolet rays cause damage to biological components such as activation of proteolytic enzymes, chain cleavage of matrix proteins, and abnormal cross-linking, and repetition of this mechanism causes skin aging that is evident in appearance.

As used herein, the term “wrinkle” refers to a state in which the skin loses its elasticity and becomes loose, for example, the skin may be folded. The term “prevention or improvement of skin wrinkles” may refer to any action that prevents or improves wrinkles by inhibiting the expression of factors related to wrinkles, or increases the total amount of collagen.

The “skin barrier strengthening” may refer to any action that enhance the function of the skin barrier, which is located on the outermost layer of the skin and prevents moisture and nutrition loss. Additionally, “impaired skin barrier function” may refer to any changes that appear in the skin due to decreased or damaged skin barrier function. For example, an impaired skin barrier function may include increased wrinkles, dryness, dermatitis, atopic dermatitis, allergic dermatitis, acne, etc.

The term “skin moisturizing” may refer to any action that maintains skin moisture or prevents moisture loss.

In an embodiment, the composition may be a cosmetic composition.

The cosmetic composition that may have, for example, an softening lotion, nourishing lotion, massage cream, nourishing cream, essence, pack, gel, ampoule, or skin-adhesive type cosmetic formulation.

The ingredients included in the cosmetic composition may include ingredients typically used in cosmetic compositions in addition to the composition as an active ingredient, and may include, for example, typical adjuvants and carriers such as stabilizers, solubilizers, vitamins, pigments, and fragrances.

In another embodiment, the composition may be a composition for external application to the skin.

The external skin preparation may be a cream, gel, ointment, skin emulsifier, skin suspension, transdermal delivery patch, drug-containing bandage, lotion, or a combination thereof. The topical preparation may be suitably formulated with ingredients commonly used in topical preparations such as cosmetics or pharmaceuticals, etc., for example, aqueous ingredients, oil-based ingredients, powder ingredients, alcohols, moisturizers, thickeners, ultraviolet absorbers, whitening agents, preservatives, antioxidants, surfactants, fragrances, colorants, various skin nutrients, or a combination thereof, according to need. The external skin preparation may also be appropriately formulated with sodium edetate, trisodium edetate, sodium citrate, sodium polyphosphate, sodium metaphosphate, metal sequestrants such as gluconic acid, etc., caffeine, tannins, bellapamil, licorice extract, glabridin, hot water extract of karin fruit, and various herbal medicines, acetic acid tocopherol, glycylitic acid, tranexamic acid and derivatives thereof or salts thereof, etc., vitamin C, magnesium ascorbate phosphate, ascorbic acid glucoside, arbutin, kojic acid, glucose, fructose, trehalose, etc., and other sugars, etc.

In another embodiment, the composition may be a health functional food composition.

The health functional food composition may be used alone or in combination with another food or food ingredient, and may be used as appropriate according to conventional methods. The amount of active ingredient mixed may be suitably determined according to the purpose of use (preventive, health or therapeutic treatment). In general, when manufacturing food or beverages, the composition of the present specification may be added in an amount of 15 parts by weight or less based on the raw materials. There is no particular limitation on the type of health functional food. Among the types of health functional foods, beverage compositions may contain various flavoring agents or natural carbohydrates as additional ingredients like regular beverages. The natural carbohydrates include monosaccharides such as glucose and fructose, disaccharides such as maltose and sucrose, polysaccharides such as dextrin and cyclodextrin, and sugar alcohols such as xylitol, sorbitol, and erythritol, etc. As a sweetener, natural sweeteners such as thaumatin and stevia extract or synthetic sweeteners such as saccharin and aspartame may be used. The health food composition may also contain nutrients, vitamins, electrolytes, flavoring agents, coloring agents, pectic acid and its salts, alginic acid and its salts, organic acids, protective colloidal thickeners, pH adjusters, stabilizers, preservatives, glycerin, alcohols, carbonation agents used in carbonated beverages, or combinations thereof. The health functional food composition may also contain pulp for the production of natural fruit juice, fruit juice beverage, vegetable beverage, or a combination thereof.

Another aspect provides a method of preventing, improving, or treating a skin condition in an individual, including the process of treating or administering an effective amount of the composition to an individual in need thereof. The specific details of the skin condition are as described above.

As used herein, the terms, “administering,” “introducing,” and “transplanting” may be used interchangeably and refer to the placement of a composition according to an embodiment into an individual by a method or route that results in at least partial localization of the composition to a desired site.

Administration may be done by methods known in the art. Administration may be administered directly to the subject by any means, such as intravenous, intramuscular, oral, transdermal, mucosal, intranasal, intratracheal or subcutaneous administration. The administration may be administered systemically or locally.

The subject may be a mammal, for example, a human, cow, horse, pig, dog, sheep, goat, or cat. The subject may be an individual in need of skin beauty improvement, for example, skin moisturizing, skin barrier strengthening, and skin wrinkle improvement effects.

The administration of the composition according to an embodiment may be from 0.1 mg to 1,000 mg per day per individual, for example as from 0.1 mg to 500 mg, from 0.1 mg to 100 mg, from 0.1 mg to 50 mg, from 0. 1 mg to 25 mg, 1 mg to 1,000 mg, 1 mg to 500 mg, 1 mg to 100 mg, 1 mg to 50 mg, 1 mg to 25 mg, 5 mg to 1,000 mg, 5 mg to 500 mg, 5 mg to 100 mg, 5 mg to 50 mg, 5 mg to 25 mg, 10 mg to 1,000 mg, 10 mg to 500 mg, 10 mg to 100 mg, 10 mg to 50 mg, or 10 mg to 25 mg. However, the dosage may vary depending on factors such as the method of formulation, mode of administration, patient age, weight, gender, medical condition, food, time of administration, route of administration, rate of excretion, and response sensitivity, and those skilled in the art may adjust the dosage accordingly to account for these factors. The number of doses may be once daily, or more than once within the range of clinically acceptable side effects, and may be administered to one or more sites, and may be administered daily or at intervals of 2 days to 5 days, for a total number of days of treatment ranging from 1 days to 30 days per treatment. If necessary, the same treatment may be repeated after an appropriate period. For animals other than humans, the same dosage per kg as for humans may be administered, or an amount equivalent to the above dosage may be administered, for example, based on the volume ratio of the organ (for example, heart) of the intended animal to a human (for example, average value).

Another aspect provides a method of preparing a subcritical water extract of plasma-treated caviar. Another aspect provides a composition for improving skin condition including the caviar subcritical water extract prepared by the above method as an active ingredient. Specific details of the skin conditions are as described above.

In an embodiment, the method may include the process of plasma-treating the lyophilized caviar; and carrying out the subcritical water extraction. In addition, before lyophilizing the caviar, the method may further include the process of degreasing the caviar, followed by hot water extraction.

### Advantageous Effects

The composition according to an aspect may be useful for preventing, improving, or treating skin-related conditions such as improving skin wrinkles, improving elasticity, and anti-aging.

### DESCRIPTION OF DRAWINGS

FIG. 1 schematically illustrates a method for preparing caviar extract according to an aspect.

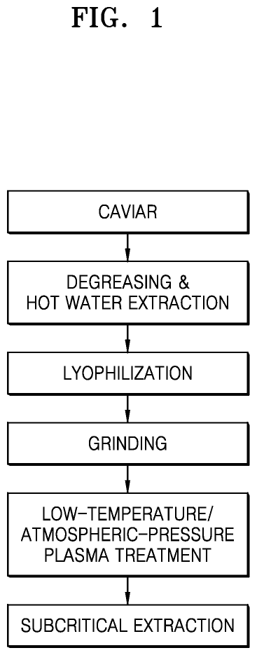

FIG. 2 is a graph showing the effect of caviar extract according to an aspect on the expression of MMP-1.

FIG. 3 is a graph showing the effect of caviar extract according to an aspect on the expression of COL1A1.

### MODE FOR INVENTION

Hereinafter, preferred examples are presented to aid understanding of the present disclosure. However, the following examples are provided only to aid understanding of the present disclosure, and the present disclosure is not limited by the following examples.

### EXAMPLE

### Example 1. Production of Caviar Low Temperature and Atmospheric-Pressure Plasma and Subcritical Water Extract

Caviar was plasma-treated under conditions of 40° C. and 5 atmospheres (atm), and was then subjected to subcritical water extraction to prepare caviar extract. Specifically, 500 g of caviar was degreased, extracted with hot water, and lyophilized. Afterwards, the lyophilized caviar was disassembled and plasma-treated under low temperature and atmospheric-pressure conditions. Plasma exposure conditions were set at a helium gas inflow rate of 5 L/min to 10 L/min, a voltage (rms) of 1 kV to 20 kV, and a distance of 5 mm to 10 mm from the source, and the plasma exposure time was 1 minute to 3 minutes for a single treatment, and the number of repeated exposures was 2 times to 10 times for 1 to 2 minutes every 30 minutes, or 2 times to 5 times for 2 minutes every hour. Thereafter, the plasma-treated caviar was subjected to subcritical water extraction under conditions of a temperature of 80° C. and 5 atmospheres (atm).

### Comparative Example

### Comparative Example 1. Preparation of Caviar Subcritical Extract

Caviar powder was prepared by lyophilizing 500 g of caviar. Afterwards, distilled water equivalent to 20 times the amount of caviar powder was added, and extraction was performed under conditions of a temperature of 80° C. and 5 atmospheres (atm) to prepare a caviar subcritical water extract.

### Comparative Example 2. Preparation of Caviar Degreased⋅Hot Water Extract

Degreased caviar was prepared by treating 50 g of lyophilized powdered caviar with supercritical carbon dioxide for 3 hours under conditions of a temperature of 40° C. to 50° C. and a pressure of 30 MPa to 31 MPa. Afterwards, distilled water equivalent to 10 times the weight of degreased caviar was added, and hot water extraction was performed for 4 hours and 30 minutes to prepare caviar degreased hot water extract.

### Experimental Example

### Anti-Aging Activity

The effects of the caviar low temperature and atmospheric-pressure plasma and subcritical water extract of Example 1 on factors related to skin aging and wrinkle formation were analyzed.

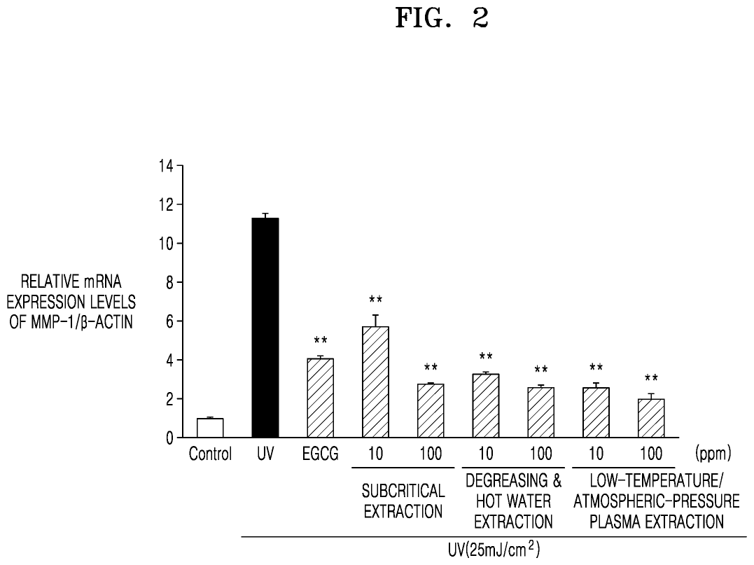

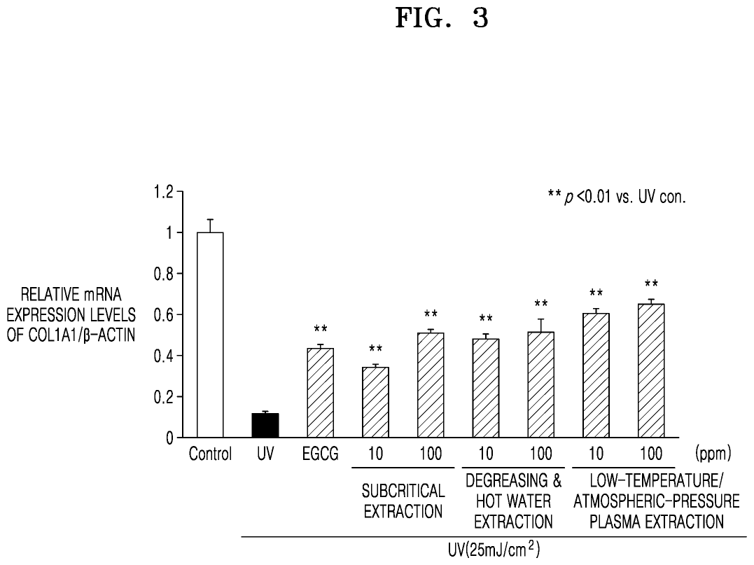

Specifically, a human fibroblast cell line (Human dermal fibroblast, Hs68) was dispensed in 6-well plates at a count of 3×10⁵ cells/well and cultured in Dulbecco's modified Eagle's medium (DMEM) containing 10% fetal bovine serum (FBS) and 1% penicillin, and the culture was performed for 24 hours in an incubator under conditions of 37° C., 5% CO₂ and 95% humidity. Afterwards, the medium was removed, DPBS was added, and UVB of 12 mJ/cm² was irradiated or not. Immediately after UVB irradiation, DPBS was removed and the medium was replaced with FBS-free medium, and Example 1 and Comparative Example 1 to Comparative Example 2 were treated at different concentrations (10 ppm, 100 ppm) and additionally cultured for 24 hours. Pure distilled water was added as a negative control group, and a group added with 1 μM EGCG was used as a positive control group. Afterwards, cells from each medium were obtained, washed with cooled PBS, RNA was isolated using an RNAprep kit (Monarch® Total RNAprep Kit, NEB, USA), RNA was quantified at 260 nm with nanodrop, and cDNA was synthesized in an amplifier (C1000 Thermal Cycler, Bio-Rad, USA) using 2 μg of RNA each. Using the synthesized cDNA as a template, Cybergreen (SYBR Green supermix, Applied Biosystems, USA) was added together with primers (see Table 1 below) and cDNA for the target genes COL1A1 and HAS2, and real-time polymerase chain reaction was performed in a real-time PCR machine (Step One Plus, Applied Biosystems, USA). The real-time PCR reaction was performed by activating the polymerase at 94° C. for 5 minutes, followed by 40 cycles of 95° C. for 30 seconds, 55° C. for 30 seconds, and 72° C. for 30 seconds. The expression levels of MMP-1 and COL1A1 genes were finally analyzed by correction for the β-actin gene, and the results are shown in FIG. 2 and FIG. 3, respectively.

TABLE 1

| Gene | Forward primer | Reverse primer |
|---|---|---|
| MMP-1 | 5′-CGAATTGCGCCGACA | 5′-GTCCCTGAACAGCCCA |
| ′ | GAGATGA-3′ | GTACTT-3′ |
| | (SEQ ID NO: 1) | (SEQ ID NO: 2) |
| COL1A1 | 5′-TGACGTTCCCATTAG | 5′-CCGTCTTTCATTACAC |
| | ACAACTG-3′ | AGGACA-3′ |
| | (SEQ ID NO: 3) | (SEQ ID NO: 4) |
| ß-actin | 5′-GGCCATCTCTTGCTC | 5′-GAGACCTTCAACACCC |
| | GAAGT-3′ | CAGC-3′ |
| | (SEQ ID NO: 5) | (SEQ ID NO: 6) |

FIG. 2 is a graph showing the effect of caviar extract according to an aspect on the expression of MMP-1.

FIG. 3 is a graph showing the effect of caviar extract according to an aspect on the expression of COL1A1.

As a result, as shown in FIG. 2, it was confirmed that Example 1 and Comparative Example 1 and Comparative Example 2 increase the expression of MMP-1 increased by ultraviolet radiation. In particular, in Example 1, it was confirmed that the expression of MMP-1 was significantly reduced compared to Comparative Example 1 and Comparative Example 2. Furthermore, as shown in FIG. 3, it was confirmed that Example 1 and Comparative Example 1 and Comparative Example 2 increase the expression of COL1A1, which was reduced by ultraviolet radiation, in a concentration-dependent manner. In particular, in the case of Example 1, it was confirmed that the expression level of COL1A1 was significantly increased compared to Comparative Example 1 and Comparative Example 2.

In other words, the caviar low temperature and atmospheric-pressure plasma and subcritical water extract according to an aspect has an excellent effect on preventing skin aging, such as increasing elasticity and inhibiting wrinkle formation, etc., by reducing the expression of MMP-1 increased by ultraviolet radiation and promoting the production of COL1A1 reduced by ultraviolet radiation. The foregoing description of the present disclosure is for illustrative purposes only, and one that has ordinary skill in the art to which the present disclosure belongs will understand that the present disclosure may be readily adapted to other specific forms without altering the technical ideas or essential features of the present disclosure. Therefore, the embodiments described above should be understood in all respects as illustrative and not restrictive.

1. A cosmetic composition for improving skin condition, comprising subcritical water extract of plasma-treated caviar as an active ingredient.

2. The cosmetic composition of claim 1, wherein the skin condition is skin aging, skin wrinkles, or skin elasticity.

3. The cosmetic composition of claim 1, wherein the plasma-treatment is performed under conditions of 10° C. to 80° C. and 0.5 atmospheres (atm) to 10 atm.

4. The cosmetic composition of claim 1, wherein the subcritical water extraction is carried out under conditions of 40° C. to 120° C. and 0.5 atm to 10 atm.

5. A method of preparing a subcritical water extract of plasma-treated caviar, comprising
plasma-treating lyophilized caviar; and
carrying out subcritical water extraction.

6. The method of claim 5, further comprising degreasing the caviar, followed by hot water extraction.

7. A cosmetic composition for improving skin condition, comprising as an active ingredient a caviar subcritical water extract prepared by the preparation method of claim 5.


SEQUENCE LISTING (6 sequences)

SEQ ID NO: 1 - DNA, 20 bp, synthetic construct
  source 1..20: mol_type = other DNA
cgaattgccgacagagatga

SEQ ID NO: 2 - DNA, 22 bp, synthetic construct
  source 1..22: mol_type = other DNA
gtccctgaacagcccagtactt

SEQ ID NO: 3 - DNA, 22 bp, synthetic construct
  source 1..22: mol_type = other DNA
tgacgttcccattagacaactg

SEQ ID NO: 4 - DNA, 22 bp, synthetic construct
  source 1..22: mol_type = other DNA
ccgtctttcattacacaggaca

SEQ ID NO: 5 - DNA, 20 bp, synthetic construct
  source 1..20: mol_type = other DNA
ggccatctcttgctcgaagt

SEQ ID NO: 6 - DNA, 20 bp, synthetic construct
  source 1..20: mol_type = other DNA
gagaccttcaacaccccagc